(19) United States
(12) Patent Application Publication
(10) Pub. No.: US 2002/0127137 A1
(43) Pub. Date: Sep. 12, 2002

(54) Solid cast air freshener and room deodorizer

(75) Inventor: William H. Scepanski, Bloomington, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: Sunburst Chemicals,Inc.

(21) Appl. No.: 09/968,697

(22) Filed: Oct. 1, 2001

Related U.S. Application Data
(60) Provisional application No. 60/236,645, filed on Sep. 29, 2000.

(51) Int. Cl. A61L 9/00

(52) U.S. Cl. 422/1; 422/4; 422/5

(57) ABSTRACT

A substantially solid cast deodorizing composition, which may include a deodorant, and optional dyes and fragrances. The deodorant and optional substances are incorporated into the present substantially solid cast composition by being mixed in a molten carrier, dispensed into a container, and allowed to solidify.

### CROSS-REFERENCES TO RELATED APPLICATIONS

This application claims priority under 35 U.S.C. §119 (e) to, and hereby incorporates by reference, U.S. Provisional Application No. 60/236,645.

### BACKGROUND

1. Field of the Invention

This invention relates to air fresheners and deodorizers and, in particular, this invention relates to air fresheners and room deodorizers in a solid cast formulation.

2. Background of the Invention

Air fresheners and deodorizers are used institutionally, as well as in private residences. When used commercially, these materials are usually available as liquids, which must be diluted before being used or which are volatized to some extent, e.g., via a wick. Liquids of this nature are also dispensed in aerosol form, usually by maintenance and cleaning personnel. During use, these liquid concentrates may be spilled, thereby hazarding the environment. These liquid concentrates may often be inadvertently and incorrectly applied in inappropriate concentrations, which either waste product or fail to solve odor problems. There then is a need for a freshener and deodorizer in a formulation not susceptible to being spilled or likely to be applied in incorrect concentrations.

### SUMMARY OF THE INVENTION

This invention substantially meets the aforementioned needs of the industry by providing a substantially solid cast composition for controlling odors. The composition may include a deodorant and a carrier. The deodorant may be substantially uniformly suspended or dissolved in the carrier. A dye, a fragrance, a viscosity modifier, and a solubility modifier may also be present in the present composition. The present invention also includes an article of manufacture, the article of manufacture including the above-referenced substantially solid cast composition solidified, and disposed, in a container.

The present invention further provides a method of making a substantially solid cast composition. The method may include melting a carrier; adding a deodorant to the melted carrier; uniformly mixing the deodorant in the melted carrier to form a substantially uniform melt (suspension and/or solution); pouring the melt into a container; and cooling the melt within the container to produce the substantially solid cast composition.

There is yet further provided a method of making a deodorizing solution. The method may include providing a substantially solid cast composition formed in a container, the container, with the substantially solid cast composition disposed therein, being inverted over a bowl, a hose depending from the bowl, the substantially solid cast composition comprising a deodorant substantially uniformly suspended or dissolved in a carrier. The method may further include impinging a quantity of water on a surface of the solid cast composition and within the container to dissolve at least a portion of the solid cast composition, thereby forming the deodorizing solution.

The present invention may still yet further provide a method for deodorizing a surface, bounded volume of air, or environment. The method may include providing the above-described deodorizing solution; and applying the deodorizing solution to the surface, bounded volume of air, or environment to eliminate or attenuate odors therein.

The present invention may further provide a method of applying a predetermined concentration of a deodorizing solution for eliminating or attenuating odors.

Additional objects, advantages, and features of various embodiments of this invention will be set forth in part in the description which follows, and in part will become apparent to those skilled in the art upon examination of the following, or may be learned by practice of the invention. The objects and advantages of various embodiments of this invention may be realized and attained by means of the instrumentalities and combinations particularly pointed out in the appended claims. Thus, these and other objects, features, and advantages of this invention will become apparent to a person of ordinary skill in the art from the description which follows.

### BRIEF DESCRIPTION OF THE DRAWING

FIG. 1 is perspective view of a dispenser of this invention, a container with the present solid cast deodorizer inverted therein.

### DETAILED DESCRIPTION OF THE INVENTION

The term “solid” is defined as an essentially homogeneous dispersion, which is nonflowable at room temperature (e.g., 68 degrees F. (20 degrees C.)). Embodiments of the solid of this invention may have a melting point of at least about 37.7 degrees C. (100 degrees F.), at least about 45 degrees C. (113 degrees F.), or at least about 52 degrees C. (125 degrees F.). A molten composition according to the present invention is placed in a container, wherein it hardens into a substantially solid cast composition. A substantially solid cast composition is one which does not exit a container when the open container is inverted (when the container opening is downwardly oriented). The present substantially solid cast composition is also distinguished from a composition which, while solid, is powdered, particulate, or granular in that it will not exit an opened, inverted container as opposed to the former products, which are free-flowing and are not consolidated in one discrete mass. In one aspect, the present substantially solid cast products have a minimum linear dimension, which is greater than about 1.27 cm, greater than about 2.0 cm, or greater than about 4 cm. Tablets, while solid, differ from the product in that tablets are made by pressing dry ingredients and an optional binder in a mold. By contrast, the present substantially solid cast compositions are formed by cooling and solidifying molten ingredients in molds. Subsequently, the present substantially solid cast composition can be dispensed from within these containers. However, the present substantially solid cast composition can be removed from the molds after hardening and then dispensed without the use of these containers as well.

The present substantially solid cast composition may include: 1) a deodorant; 2) a carrier; 3) an air freshener; and 4) optional ingredients to, e.g., modify the viscosity of the melt or modify the solubility of the use solution.

The present substantially solid cast may include fragrances, which are pleasant smelling and which mask objectionable odors.

In contrast to air fresheners, deodorants are contemplated to include substances which chemically alter odor producing substances. Non-limiting examples of deodorants include:

1. Bacterial spores, e.g., Sprozyme BCC™ (Semco Labs).

2. Enzymes, e.g., Vegetable Protein Concentrate A560™ (Carruba, Inc.).

3. Organic zinc compounds, e.g.,

Zinc ricinoleate, e.g., Tegosorb™ (Goldschmidt, AG),

Zinc alkyl sulfates,

Zinc alkylsulfonates,

Zinc alkylarylsulfates, and

Zinc alkylarylsulfonates.

4. Peroxides, e.g., hydrogen peroxide.

5. Perborates, e.g., sodium perborate.

6. Percarbonates, e.g., sodium percarbonate.

7. Persulfates, e.g., sodium persulfate.

8. Organic peroxides, e.g.,

Benzoyl peroxide,

Dicumyl peroxide, and

Di (2-tert-2-butyl peroxyisopropyl) benzene.

9. Organic peroxyacids (RCO₃H), e.g.,

Peroxyformic acid,

Peroxyacetic acid,

Peroxybenzoic acid, and

Metachloroperoxybenzoic acid.

10. Meelium™ (Prentiss Drug and Chemical Co.).

11. Cationic surfactants, e.g., quaternary ammonium salts, such as,

Alkyldimethyl ammonium chloride,

Alkyldimethylbenzyl ammonium chloride, and

Alkyldimethylethylbenzyl amnonium chloride

These and other deodorants may be present in the substantially solid cast composition of this invention in an effective amount or in an amount between about 0.01% and 90%, between about 0.1% and 40%, between about 0.1% and 20%, between about 1% and 10%, or any range subsumed therein, by weight.

The present invention also includes one or more carriers to incorporate the other substances into the present solid cast mass. It is also contemplated that at least some of the above-described deodorants may also function as carriers. These carrier materials may all be solid at a temperature of about 38 degrees C. (100 degrees F.). However, under some circumstances, it may be desirable to include carriers with melting points of about 45 degrees C. (113 degrees F.) or about 52 degrees C. (125 degrees F.). Non-limiting examples of these carriers include compounds solid at one of the preceding temperatures from one or more of the following groups:

1. Polyethylene glycols.

2. Linear or branched alkoxylates, e.g., ethoxylates, propoxylates.

3. Linear or branched alkylaryl alkoxylates, e.g., linear or branched alkylaryl ethoxylates, linear or branched alkylaryl propoxylates.

4. Fatty acid alkoxylates, e.g., fatty acid ethoxylates, fatty acid propoxylates.

5. Ethylene oxide/propylene oxide (EO/PO) polymers.

6. Fatty alkanolamides.

7. Fatty acids.

8. Fatty alcohols.

9. Sorbitan fatty esters.

10. Glyceryl mono- and diesters.

11. Polyethylene glycol mono- and diesters.

12. Phosphate esters.

13. Fatty amines and diamines.

14. Urea

15. Quaternary ammonium compounds.

16. Hydrated salts of alkali metal or ammonium phosphates.

17. Hydrated salts of alkali metal or ammonium carbonates.

18. Hydrated salts of alkali metal or ammonium sulfates.

19. Hydrated salts of alkali metal or ammonium silicates.

20. Hydrated salts of alkali metal or ammonium borates.

These carriers may be present in amounts, by weight, of between about 10% and 99.9%, between about 40% and 99.9%, between about 60% and 90%, between about 60% and 80%, or any range subsumed therein. While some carriers are considered to be inert, other carriers may function as solubility modifiers under certain circumstances, for example, as emulsifying agents or surfactants which synergistically assist the use solution in penetrating water-resistant surfaces so that the odor mitigating substance can contact the odor-producing substance.

The present invention also contemplates including fragrances. Non-limiting examples of these fragrances include:

1. Irish Spring™ fragrance (Intercontinental Fragrances).

2. Lemon fragrance (Value Products).

3. 9501 Nonacid Bathroom Cleaner Fragrance™ (Intercontinental Fragrances).

These fragrances may be present in amounts, by weight, of between about 0.001% and 20%, between about 0.01% and 15%, between about 0.1% and 10%, or any range subsumed therein. A person of ordinary skill in the art will appreciate that other suitable fragrances are readily available and may be utilized in the present invention without undue experimentation.

Optional ingredients are also contemplated to be within the scope of the present invention. These optional ingredients may include dyes such as (e.g., Pylakor Dark Violet Dye (Pylam Products)), mineral oil, water, alcohols, or glycols. In some embodiments, the optional ingredients are also synergistically present to alter the solubility of the use solution in penetrating water resistant surfaces as described above. Optional ingredients may also be used to adjust the viscosity of the melt before pouring the melt into a container.

The present solid cast composition may be made by melting the carrier in a mixer or vat. In some cases, it is desirable to raise the temperature of the melted carriers\ significantly above the melting point until a desired viscosity is attained. The deodorant is then added to the melted carrier and is thoroughly mixed to a substantial degree of uniformity. The melt with the carrier and deodorant may then allowed to cool to a desired degree of viscosity. The liquid mixture is then poured into a container and allowed to solidify as it cools further. These containers include plastic jars, e.g., 8-128 fluid ounce volumes. Alternatively, the liquid mixture is poured into molds and allowed to solidify.

Thus, the present invention includes a substantially solid cast composition, which is substantially homogeneous on the scale of quantities used in any typical application. Stated otherwise, while the substantially solid cast composition may not be a homogeneous solid mixture on a microscopic level, any granules or undissolved particles will be dispersed to form an effectively uniform solution/suspension for any practical application.

The present substantially solid cast composition can also be formed by being poured into molds, then removed from the molds before being used. Thus, the present solid cast composition can be used with or without being present in containers. For example, the present substantially solid cast composition can be formed in pre-measured quantities, which can then be dissolved in water to form a use solution for appropriate applications. However, the substantially solid cast composition, dispensed without a container, may not be the preferred product formulation under some circumstances because the present cast generally dissolves slowly and often provides an irregular or a nonuniform series of sequential use solution concentrations. It may be more preferable to form the substantially solid cast composition in a container which can be used in an appropriate dispensers. These dispensers may also facilitate dissolving of the product and dispensing of the deodorizing solution.

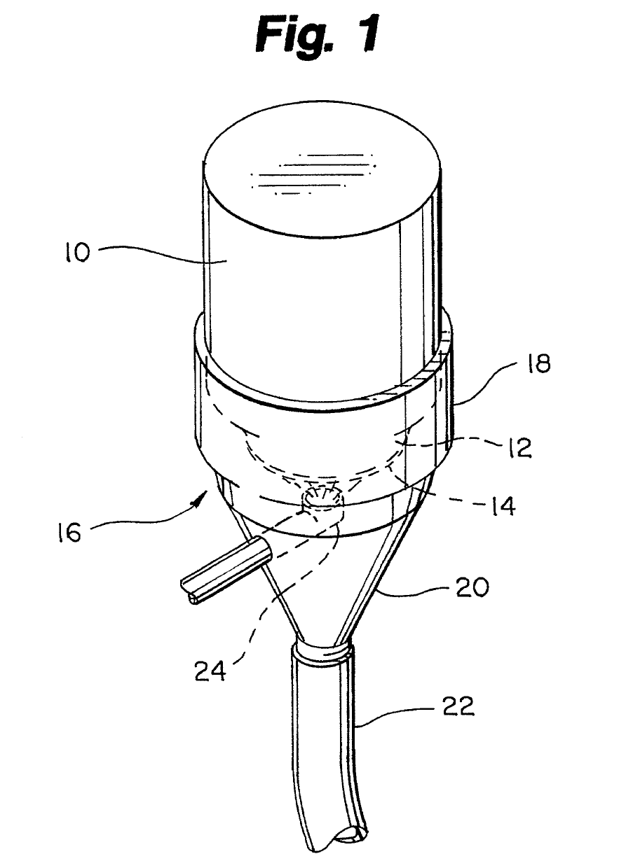

Typically, and as disclosed in FIG. 1, a container 10 will be generally cylindrical, with a tapered portion 12 ending at an opening 14. A dispenser 16 may have a bowl 18, into which the container 10 is inverted during use. A bottom 20 of the bowl 18 has a funnel shape to direct solutions into a length of tubing 22 attached at the end of the funnel shaped bottom 20 of the bowl 18. A spray tip 24 connected to a source of pressurized water projects up into the funnel portion 20 of the bowl 18. The spray tip 24 is placed such that it sprays water into the container 10 to dissolve a portion of the present substantially solid cast composition, which is both formed and dispensed within the container 10. The funnel 20 directs the solution into the tubing 22. The solution may be further diluted before being used as well.

Specific examples of suitable dispensers are described in U.S. Pat. Nos. 5,342,587, 5,478,537, 5,549,875, 5,435,157, and 5,846,499, each hereby incorporated by reference. In some of these dispensers, a water inlet is attached to a fitting with a secondary outlet that directs a portion of water to a spray tip within the dispenser bowl. In one embodiment, the primary outlet of the fitting drains into an outlet to a Y-fitting which has a second inlet connected to the funnel at the end of the bowl. The outlet to the Y-fitting can be connected to the end of a hose. Using this dispenser, the present deodorizing composition, stored as a substantially solid composition, can be dissolved, and the use solution selectively added to a stream of water directed out the end of a hose. This is particularly convenient for deodorizing large areas which can be hosed down with the deodorizer dispensed from the end of the hose. For smaller applications, the hose can be directed into a bucket, spray or aerosol applicator, or other applicator. Other designs for dispensers can also be used.

By way of illustration, and not limitation, the following illustrative examples of the present substantially solid cast are disclosed.

| Example 1. | |
|---|---|
| Polyethylene glycol (mw8000) | 90% |
| Sporzyme BCC ™ | 2% |
| Lemon fragrance | 8% |
| Example 2. | |
| Polyethylene glycol (MW8000) | 90% |
| Vegetable Protein Extract (Carruba) | 5% |
| Lemon Fragrance | 5% |
| Example 3. | |
| Nonylphenol (30 mole) ethoxylate | 85.995% |
| Zinc ricinoleate | 4.000% |
| Irish Spring Fragrance | 10.000% |
| Violet dye | 0.005% |
| Example 4. | |
| Linear alcohol (100 mole) ethoxylate | 84% |
| Meelium ™ | 10% |
| 9501 Non Acid Bathroom Cleaner Fragrance ™ | 6% |
| Example 5. | |
| Lauric monoethanolamide | 90.0% |
| Zinc ricinoleate | 10.0% |
| Example 6. | |
| Urea | 81% |
| Water | 5% |
| Zinc ricinoleate | 4% |
| 9501 Non Acid Bathroom Cleaner Fragrance ™ | 10%. |
| Example 7. | |
| Lauric monoethanolamide | 90% |
| Lemon fragrance | 10% |
| Example 8. | |
| Nonylphenol (100 mole) ethoxylate | 88% |
| Alkyldimethylbenzyl ammonium chloride | 10% |
| Lemon fragrance | 2% |
| Example 9. | |
| Nonylphenol (30 mole) ethoxylate | 80% |
| Sodium perborate | 20% |

Because numerous modifications of this invention may be made without departing from spirit thereof, the scope of the invention is not to be limited to the embodiments illustrated and described. Rather, the scope of the invention is to be determined by the appended claims and their equivalence. Although the present invention has been described with reference to preferred embodiments, workers of ordinary skill in the art will recognize that various modifications and changes may be made without departing from the spirit and the scope of the invention.

What is claimed is:

1. A substantially homogeneous solid cast deodorizing composition, comprising:
a deodorant; and
a carrier, the deodorant substantially uniformly suspended or dissolved in the carrier.

2. The composition of claim 1, further comprising an additional ingredient selected from a dye, and a fragrance.

3. The composition of claim 1, in which the carrier functions as a solubility modifier.

4. The composition of claim 1, in which the carrier functions as a viscosity modifier.

5. The composition of claim 1, the deodorant selected from a bacterial spore, an enzyme, an organic zinc compound, a peroxide, a perborate, a percarbonate, a persulfate, an organic peroxide, an organic peroxyacid, a cationic surfactant, and any combination thereof.

6. The composition of claim 1, the deodorant comprising a peroxide.

7. The composition of claim 1, the deodorant comprising a bacterial spore.

8. The composition of claim 1, the deodorant comprising an enzyme.

9. The composition of claim 1, the deodorant comprising a quaternary ammonium salt.

10. The composition of claim 1, the deodorant comprising an organic zinc compound.

11. The composition of claim 10, the organic zinc compound selected from a zinc ricinoleate, a zinc soap of a fatty acid, a zinc alkylsulfate, a zinc alkylsulfonate, a zinc alkylarylsulfate, and a zinc alkylarylsulfonate.

12. The composition of claim 1, the deodorant present in an amount between about 0.01% and 90%, by weight.

13. The composition of claim 1, the deodorant present in an amount between about 0.1% and 40%, by weight.

14. The composition of claim 1, the deodorant present in an amount between about 1% and 10%, by weight.

15. The composition of claim 1, the carrier present in an amount between about 10% and 99.9%, by weight.

16. The composition of claim 1, the carrier present in an amount between about 60% and 90%, by weight.

17. The composition of claim 1, the carrier present in an amount between about 60% and 80%.

18. An article of manufacture, comprising:
a container; and
the composition of claim 1, solidified in the container.

19. A method of making a substantially solid cast deodorizing composition, comprising:
melting a carrier;
adding a first deodorant to the melted carrier;
uniformly mixing the first deodorant in the melted carrier to form a substantially uniform melt;
pouring the melt into a container; and
cooling the melt to produce the substantially solid cast composition.

20. The method of claim 19, wherein, the carrier is a second deodorant.

21. A method for making a deodorizing solution, comprising:
providing a solid cast composition formed in a first container, the first container and solid cast composition inverted over a bowl, a hose depending from the bowl, the solid cast composition comprising a deodorant substantially uniformly dispersed in a carrier; and
impinging a quantity of water on a surface of the solid cast composition to dissolve at least a portion thereof, thereby forming the deodorizing solution.

22. The method of claim 21, further comprising draining the deodorizing solution from the bowl through the hose to a second container.

23. The method of claim 21, further comprising diluting the deodorizing solution.

24. A method for deodorizing a surface, comprising:
providing the solution of claim 21; and
applying the solution to the surface.

25. The method of claim 24, in which the solution is applied to a floor or a wall.

26. The method of claim 24, in which the solution is applied by immersing a surface in the solution.

27. The method of claim 24, in which the solution is applied to a trash disposal container.

28. The method of claim 24, in which the solution is applied to a surface on a portable toilet.

29. The method of claim 24, in which the solution is applied to a bathroom surface.

30. The method of claim 24, in which the solution is applied to a carpet.

31. A method of deodorizing a volume of air, comprising:
dissolving a portion of a substantially homogeneous solid cast composition including a carrier and a deodorant uniformly dispersed in the carrier, thereby forming a use solution; and
exposing the use solution to a volume of air.

32. The method of claim 31, in which exposing the use solution to a volume of air includes forming a mist with the use solution.

33. The method of claim 31, in which exposing the use solution to a volume of air includes forming an aerosol with the use solution.

34. The method of claim 31, in which the volume of air is disposed in a bathroom, the use solution exposed to the volume of air in the bathroom.
